(19) United States
(12) Patent Application Publication
(10) Pub. No.: US 2022/0340464 A1
(43) Pub. Date: Oct. 27, 2022

(54) System and Method of Increasing Methane Production in Anaerobic Digesters

(71) Applicant: NCH Corporation, Irving, TX (US)

(72) Inventors: Gabriel F.K. Everett, York, SC (US); Charles J. Greenwald, Irving, TX (US); Judith G. Pruitt, Mesquite, TX (US)

(21) Appl. No.: 17/726,913

(22) Filed: Apr. 22, 2022

Related U.S. Application Data
(60) Provisional application No. 63/179,836, filed on Apr. 26, 2021.

(51) Int. Cl. C02F 3/34; C12N 1/20; A01N 63/22

(52) U.S. Cl. CPC C02F 3/348; C12N 1/20; A01N 63/22

(57) ABSTRACT

A spore germination composition and method to produce a bioaugmentation solution that is added to an anaerobic digester or partially aerobic digester to increase biogas production. A nutrient-germinant composition comprises L-amino acids a phosphate buffer, an industrial preservative, and an optional source of potassium. The composition and spores of one or more Bacillus species are heated to a preferred elevated temperature range of 35° C. to 60° C. for an incubation period of around 20 to 60 minutes to form a bioaugmentation solution that is dispensed to the digester, preferably to the hydrolysis stage of the digester. A dose of bioaugmentation solution is added to the digester around once per day in an amount to provide at least 1000 CFU per mL of the full volume capacity of the digester, which can increase methane production by around 5 to 10% over operation of the digester without the bioaugmentation solution.

### CROSS-REFERENCE TO RELATED APPLICATION

This application claims the benefit of U.S. Provisional Application Ser. No. 63/179,836 filed on Apr. 26, 2021.

### BACKGROUND OF THE INVENTION

### 1. Field of the Invention

The present invention relates to a nutrient-germinant and spore composition and a point-of-use incubation method of germinating the bacterial spores for the purpose of increasing methane production in anaerobic digesters and bioaugmentation of water collection systems, such as lift stations and grease interceptors.

### 2. Description of Related Art

Spore germination is a multistep process in which spores are revived from a dormant state to a vegetative growth state. The first step is one by which spores are activated and are induced to germinate, typically by an environmental signal called a germinant. This signal can be a nutrient such as an L-amino acid. Nutrient germinants bind to receptors in the inner-membrane of the spore to initiate germination. Additionally, sugars have been shown to increase the binding affinity of L-amino acids for their cognate receptors. The second phase of germination is an outgrowth step in which the spore's metabolic, biosynthetic, and DNA replication/repair pathways initiate. After the outgrowth step, spore revival is complete and cells are considered to be in a vegetatively growing state.

It is known that spores can be induced to germinate via heat-activation in the presence of nutrient germinants (U.S. patent Ser. No. 10/610,552). Specifically, it is known that concentrated Bacillus spores can be combined with a concentrated nutrient-germinant solution at an elevated temperature (e.g. 41-44° C.) to induce germination. These spores can be added to wastewater systems for the purpose of bioaugmentation—the use of microorganisms (e.g. bacteria) to speed up the rate of degradation of a contaminant. In particular, bioaugmentation is useful in systems with large concentrations of contaminants (e.g. fat, oil, grease, sugar, starch, etc.) like anaerobic digesters. Anaerobic digesters use bacteria to break up contaminants in the absence of oxygen. Anaerobic digestion is ideal for high organic waste including solid food waste, manufacturing process water, agricultural waste, and residual sludge from wastewater treatment processing. These waste byproducts would otherwise be incinerated or put into landfills. The anaerobic digestion of waste produces gas (methane or biomethane) that can be harvested and used as an energy source.

Anaerobic digestion is a complex process requiring several steps. First, hydrolytic bacteria break down large polymers (e.g. carbohydrates, protein, and fats) into their constituent molecules (simple sugars, amino acids, and fatty acids, respectively). The fatty acids produced in the first step include long and short chain fatty acids. Second, acidogenic bacteria convert the smaller constituent molecules into ammonia, carbon dioxide, hydrogen, and volatile fatty acids. Third, acetogenic bacteria convert volatile fatty acids into acetic acid and other compounds including hydrogen and carbon dioxide. In the fourth step, methanogenic bacteria convert those molecules into carbon dioxide and methane gas. This gas is often collected for use as an energy source. Therefore, efficient bioaugmentation of an anaerobic digester results in an increase of methane gas production. Typically, an anaerobic digester can produce around 1 to 20 kg of biogas, containing 50 to 70% methane, per tonne of feed stock with an average production of 0.1 to more than 3 MWe annually. For example, U.S. Pat. No. 6,299,774 discloses digestion in a single anaerobic digester using Clostridium that is capable of producing around 5-8 ft³ or methane per pound of animal waste feedstock, with the methane having an average rating of about 500-800 BTU.

It is also known to use various systems and methods to enhance methane production. For example, operating the digester at medium to high temperatures increases biogas production over lower temperatures; two-phase processes, where the acid phase and methanogenic phase are separate, may increase biogas production; and bioaugmentation may to increase biogas production in anaerobic digesters. Bioaugmentation generally refers to intentionally and separately adding cultured or grown bacteria to a digester (beyond the initial addition to start the digester) to augment the effectiveness of the native bacteria in the digester (the bacteria that is initially added, along with bacteria found in feedstocks to the digester, both of which may propagate in the digester). For example, U.S. Pat. No. 9,074,179 discusses bioaugmentation with cellulose degrading bacteria that may increase methane production in a digester fed with animal manure by as much as 93%, but notes that the increase is only temporary after inoculation due to problems with washout and competition by the native microorganisms. The '179 patent also discloses bioaugmentation with methanogens that are cultured in the presence of oxygen at specified levels and then added to an aerobic digester at a preferred rate of at least 10 mg VSS/L-day to increase methane production by around 60% compared to a non-bioaugmented control digester.

There is a need for a rapid spore incubation and activation method that will allow generation of active Bacillus species in a single step at a point-of-use location where the bacteria will be distributed to an anaerobic digester for the purpose of bioaugmentation and consequent increase in methane production. Accordingly, this invention describes a simple method for spore germination using a nutrient germinant concentrate simultaneously with heat incubation in a single step.

### SUMMARY OF THE INVENTION

According to one preferred embodiment, a nutrient-spore composition comprising a nutrient germinant composition and certain anaerobic or facultative anaerobic bacteria is heated to a temperature above ambient temperature at or near an aerobic digester for a period of time (an incubation period) to form a bioaugmentation solution that is then added to the first (hydrolysis) step of anaerobic digestion system or digester. It is preferred to use Bacillus to form the bioaugmentation solution, which results in increased substrates for the next step in the anaerobic digestion process (acidogenesis). In addition, any acetate produced in the first step can be consumed by methanogenic bacteria in the fourth step (methanogenesis). The bioaugmentation solution is particularly well suited for use with a single-stage digestion system, double-stage digestion system, and lagoon systems.

A nutrient-germinant composition according to one preferred embodiment of the invention comprises one or a combination of many L-amino acids, optionally D-glucose or D-fructose (which increases the binding affinity of L-amino acids for their cognate receptors in the spore coat), and a pH neutral buffer (such as a phosphate buffer, Tris buffer, or HEPES buffer). According to another preferred embodiment, a nutrient-germinant composition further comprises an industrial preservative, such as the commercially available Kathon/Lingard CG (which has active ingredients comprising methyl chloro isothiazolinone and methyl isothiazolinone). An industrial preservative is particularly preferred when the nutrient-germinant composition is premixed with a spore composition into a premixed nutrient-spore composition as further described herein, but may be omitted if the bacteria are separate from the nutrient-germinant composition and only mixed together at the point-of-use and heating. The inclusion of a general, industrial preservative in the nutrient-germinant or premixed nutrient-spore composition aids in long-term storage and/or germination inhibition, which is particularly useful when the composition is in the preferred concentrated form. Even when not premixed with spores, the inclusion of an industrial preservative aids in preventing any background contamination from growing in the nutrient-germinant composition during storage. According to another preferred embodiment, the nutrient-germinant composition also comprises a source of potassium ions, such as potassium chloride or monopotassium phosphate or dipotassium phosphate. A source of potassium is optional and depends on the bacteria species to be used with the nutrient-germinant composition. The potassium phosphate (mono- or di-) may act as both a source of potassium and a buffer and can replace the need for potassium chloride and a separate buffer. According to another preferred embodiment, the nutrient-germinant composition includes both D-glucose and D-fructose. According to another preferred embodiment, the composition does not include any added sugars, such as D-glucose or D-fructose.

According to another preferred embodiment, a nutrient germinant composition according to the invention is in concentrated form and is diluted to 0.01% to 10% strength in water or another diluent at the point-of-use, more preferably around 1 to 5% strength, and most preferably around 2 to 4% strength. Most preferably, the concentrated composition is in a liquid form, which is easier and faster to mix with diluent at the point-of-use, but solid forms such as pellets or bricks or powder may also be used.

Due to the unique, anaerobic nature of these digesters, special bacteria must be used to accomplish hydrolytic bioaugmentation. Specifically, the bacteria must be anaerobic or facultative anaerobes. According to one preferred embodiment, bacteria to make a bioaugmentation solution comprise the following Bacillus species, but other anaerobic bacteria or facultative anaerobes may also be used: algicola, amyloliquefaciens, arseniciselenatis, barbaricus, circulans, coagulans, firmus, jeotgali, krulwichiae, licheniformis, mycoides, mojavensis, nealsonii, novalis, pseudomycoides, safensis, simplex, smithii, sonorensis, subtilis, thermoamylovorans, vedderi, and vallismortis. Most preferably, the Bacillus species are in spore form prior to heating for the incubation period.

According to another preferred embodiment, aerobic Bacillus bacteria may be used for a bioaugmentation solution for use with digester systems with low oxygen concentrations (not fully anaerobic). Preferred species include, but are not limited to clausii, lactis, laterosporus, laevolacticus, lentus, polymyxa, pumilus, megaterium, sphaericus, and toyonensis. Most preferably, the Bacillus species are in spore form prior to heating for the incubation period.

The bacteria used to make a bioaugmentation solution are preferably in a spore composition. According to one preferred embodiment, a spore composition in powdered form comprises spores of one or more bacteria species, preferably Bacillus species and most preferably one or more of the specific species described herein, but other bacteria may also be used. Most preferably, the spore composition comprises bacterial spores that are in a dry, powder blend of 40-60% salt (table salt) and 60-40% bacteria spores. According to another preferred embodiment, a spore composition in liquid form comprises Bacillus spores, one or more surfactants to disperse the spores, a thickener to suspend the spores in solution, acid (or salts of acids) for pH adjustment, and an industrial preservative to extend shelf life and prevent contamination. Thickeners and acids/salts may include those disclosed in U.S. Pat. No. 10,653,729, which is incorporated by reference. Other acids, such as of phosphoric acid, acetic acid, hydrochloric acid, or salts of these acids, may also be used. Most preferably, the surfactants comprise polysorbate 80, or an amphoteric surfactant, preferably one comprising cocamidopropyl betaine, or a combination thereof. Thickeners preferably comprise xanthan gum.

According to another preferred embodiment, a spore composition comprises around 75-125 g/L (approx. 2×10¹¹ CFUs/g) of a Bacillus spore blend (preferably comprising 40-60% salt (table salt) and 60-40% bacteria spores), (2) around 1.5-2.5 g/L Tween 80 (or polysorbate 80, a surfactant), (3) around 1.5-2.5 g/L of Amphosol CG (an amphoteric surfactant comprising around 30% active cocamidopropyl betaine), (4) around 2.175-3.625 g/L of Keltrol (a thickener comprising xanthan gum), (5) around 0.75-1.25 g/L citric acid, and (6) 0.75-1.25 g/L of Linguard ICP (industrial preservative). Most preferably, the pH of the spore composition is around 4.3-5.5, most preferably around 4.5+/−0.2. The amount of Bacillus spore blend preferably comprises at least around 2×10¹¹ CFUs/g of the bacteria.

According to one preferred embodiment, a nutrient-germinant composition and a spore composition are separate and mixed together at the time of heating at or near the point-of-use.

According to another preferred embodiment, a nutrient-germinant composition and a spore composition are premixed in a nutrient-spore composition prior to delivery to the point-of-use or point-of-sale. According to another preferred embodiment, a nutrient-spore composition according to the invention is in concentrated form and is diluted to 0.01% to 10% strength in water or another diluent at the point-of-use, more preferably around 1 to 8% strength, and most preferably around 2 to 4% strength. Most preferably, the concentrated composition is in a liquid form, which is easier and faster to mix with diluent at the point-of-use, but solid forms such as pellets or bricks or powder may also be used.

According to another preferred embodiment, a premixed nutrient-spore composition comprises spores of one or more Bacillus species in combination with any of the nutrient-germinant composition ingredients described herein and (1) an industrial preservative, (2) a germination inhibitor, such as NaCl or D-alanine, and/or (3) the premixed nutrient-spore composition comprises buffers (such as those listed herein for a nutrient-germinant composition) and/or one or more acids or salts of acids to lower the pH of the composition. The spores in the premixed nutrient-spore composition are kept in a dormant spore-state by the use of an industrial preservative, germination inhibitor, a low pH, or combination thereof until the composition is diluted at the point of use. This dilution can be a diluent used to dilute the premixed nutrient-spore composition if in a concentrated form, water in the anaerobic digester, or water in other water collection system to which the bioaugmentation solution is added (such as lift station or grease interceptor/trap or in a drain pipe). According to another preferred embodiment, a premixed nutrient-spore composition has a pH of around 2 to 9, more preferably 3 to 7, and most preferably 4 to 5, to maintain the spores in spore form and prevent germination during storage and prior to the time of use. As an alternative to the buffers described herein, the premixed nutrient-spore composition comprises one or more acids or salts of acids to achieve a pH in these preferred ranges. Preferred acids or salts of acids comprise one or more of phosphoric acid, acetic acid, hydrochloric acid, or citric acid.

In another preferred embodiment, the present invention comprises a method of germinating spores of Bacillus species using a nutrient germinant composition at an elevated temperature; preferably in a range of 35-60° C., more preferably in the range of 38-50° C., and most preferably in the range of 41° C. to 44° C. for a period of time (an incubation period). In other preferred embodiments, the elevated temperature range is any individual temperature or sub-range between 35-60° C., including a sub-range that overlaps one of the previously mentioned sub-ranges. The incubation period preferably ranges from 15-60 minutes, more preferably around 20-60 minutes. At the end of the incubation period, a bioaugmentation solution is formed and is dispensed to the point-of-use, such as an anaerobic digester. A bioaugmentation solution preferably comprises primarily activated or metastable state bacteria, that will undergo an outgrowth phase to become fully vegetative after being dosed to the anaerobic digester; although a long incubation period may be used to provide fully vegetative bacteria if desired.

Most preferably, a nutrient-germinant composition in concentrated form according to a preferred composition of the invention is used in the incubation methods of the invention, but other nutrient-germinant compositions may also be used. Preferably, the incubation method is carried out at or near the point-of-use—the site or near the site where the germinated spores will be used (such as in an anaerobic digester) and further comprises dispensing the germinated spores to the point-of-use. Preferred methods according to the invention may be carried out in any incubation device that has a reservoir capable of holding a volume of spores (preferably a spore composition according to a preferred embodiment of the invention), liquid (typically water), nutrient-germinant composition (preferably one according to a preferred embodiment of the invention, or a premixed nutrient-spore composition according to a preferred embodiment of the invention) and that is capable of heating the mixture during an incubation period. Most preferably, the methods are carried out in a device that is also capable of mixing those ingredients, automatically shutting-off heating at the end of the incubation period, and automatically dispensing a bioaugmentation solution (or a probiotic or treatment solution) comprising the spores to a point-of-use. Preferred methods may also be carried out as a batch process or as a continuous process. Any variety of spore forms, such as dried powder form, a liquid suspension, or a reconstituted aqueous mixture, may be used with the preferred methods of the invention.

The preferred embodiments of the invention have broad utility and application and will allow for rapid germination of spores of Bacillus species at a point-of-use. The preferred embodiments are particularly useful in preparing spores for use in bioaugmentation of an anaerobic digester, lift station, grease interceptor, industrial process water plant, or drain pipe. When a bioaugmentation solution is added to a digester according to preferred methods of the invention, methane production may be increased by around 1 to 10%, preferably at least 5%, compared to the operation of the digester without the bioaugmentation solution. Feeding a bioaugmentation solution according to a preferred embodiment of the invention comprising around 1×10¹³ CFU of bacteria per day with the feed material (approx. 165 tonnes daily) resulted in an average increase in methane production of 5-7% compared to the production of the digester before treatment.

### DESCRIPTION OF THE PREFERRED EMBODIMENTS

A nutrient-spore composition according to a preferred embodiment comprises a nutrient-germinant composition and a spore composition. The nutrient-spore composition may be premixed and preferably in a concentrated form prior to shipment for end use or its ingredients may be mixed together at or near the end use sites.

A nutrient-germinant composition according to one preferred embodiment of the invention comprises one or more L-amino acids, D-glucose (which increases the binding affinity of L-amino acids for their cognate receptors in the spore coat and is optional), D-Fructose (optional, depending on bacteria species), an optional buffer to provide the proper pH for spore germination (such as HEPES sodium salt, a phosphate buffer, or a Tris buffer), an optional source of potassium ions (such as KCl), and an industrial preservative. In another preferred embodiment, the composition comprises both D-glucose and D-fructose. The use of D-fructose, a combination of D-glucose and D-fructose, and a potassium ion source are dependent on the species of bacteria as will be understood by those of ordinary skill in the art. According to another preferred embodiment, a nutrient-germinant composition does not include any added sugars, such as D-fructose or D-glucose. It is preferred to use a preservative that is pH compatible with the composition, which has a relatively neutral pH.

According to another preferred embodiment, the nutrient-germinant composition is premixed with spores of one or more Bacillus species (or a spore composition) prior to delivery to the point-of-use or point-of-sale into a premixed nutrient-spore composition. Most preferably, a premixed nutrient-spore composition comprises one or more germination inhibitors. In the case of a combined, premixed nutrient-spore composition containing nutrient germinants and Bacillus spores, a low pH (preferably a pH of 2-6, more preferably 3.5 to 5.5, most preferably 4 to 4.5) may be combined with an industrial preservative to prevent premature germination of the spores. Alternatively, spores (preferably in a spore composition) may be separately added to the nutrient-germinant composition according to the invention at the point-of-use to form a nutrient-spore composition. According to another preferred embodiment, the nutrient-germinant composition or premixed nutrient-spore composition is in a concentrated form, most preferably as a concentrated liquid, and is diluted at the point-of-use.

According to other preferred embodiments, a nutrient germinant composition or nutrient-spore composition does not include any (1) sources of nitrogen-hydrogen compounds (such as ammonia or NH₂Cl), (2) chloride compounds other than sodium chloride or potassium chloride (such as nickel (II) chloride, calcium chloride, or NH₂Cl) and/or (3) sugars (such as D-fructose or D-glucose).

Preferred L-amino acids include L-alanine, L-asparagine, L-valine, and L-cysteine. In a further embodiment of the concentrate composition, L-amino acids can be provided as a hydrolysate of soy protein. When in a concentrated form, the nutrient-germinant composition preferably comprises a solution of one or more of the above mentioned L-amino acids in the weight range of 8.9-133.5 g/L, more preferably 13.2-111.25 g/L, and most preferably 17.8-89 g/L each; D-glucose (optional) and/or D-fructose (optional) in the weight range of 18-54 g/L, more preferably 27-45 g/L, and most preferably 30-40 g/L each; KCl (optional, as a source of potassium) in the weight range of 7.4-22.2 g/L, more preferably 11.1-18.5 g/L, and most preferably 14-16 g/L; monosodium phosphate in a weight range of 10-36 g/L, more preferably 15-30 g/L, and most preferably 20-24 g/L; disodium phosphate in a weight range of 30-90 g/L, more preferably 21.3-75 g/L, and most preferably 28.4-60 g/L; and an one or more industrial preservatives at a final (total) weight range of 0.8-3.3 g/L, more preferably 1.2-2.7 g/L, most preferably 1.6-2.2 g/L. In addition to or in place of the monosodium/disodium phosphate buffer, the composition may comprise Tris base in a weight range of 15-61 g/L, more preferably 24-43 g/L, and most preferably 27-33 g/L; or HEPES buffer in a weight range of 32.5 97.5 g/L, more preferably 48.75-81.25 g/L, and most preferably 60-70 g/L. Optionally, monopotassium phosphate may also be used as a source of potassium ions, preferably in a weight range of 13.6-40.8 g/L, more preferably 20.4-34 g/L, and most preferably 26-29 g/L. Optionally, dipotassium phosphate may also be used as a source of potassium ions, preferably in a weight range of 8.7-26.1 g/L, more preferably 13-21.75 g/L, and most preferably 16-19 g/L. The amounts of these ingredients are important aspects of the invention because higher concentrations would render some ingredients insoluble and lower concentrations would be ineffective at germinating spores.

According to other preferred embodiments, a nutrient-spore composition or a nutrient-germinant composition comprises one of more of the following ingredients: L-alanine, potassium chloride, disodium phosphate, monosodium phosphate, and an industrial preservative (preferably comprising methyl chloro isothiazolinone and/or methyl isothiazolinone). According to other preferred embodiments, a nutrient-spore composition or a spore composition comprises one or more of the following ingredients: a Bacillus spore blend (preferably 40-60% salt (table salt) and 60-40% Bacillus spores), polysorbate 80, an amphoteric surfactant (preferably comprising cocamidopropyl betaine), a thickener (preferably xanthan gum), citric acid, and an industrial preservative (if not already included, preferably comprising methyl chloro isothiazolinone and/or methyl isothiazolinone). According to other preferred embodiments, a nutrient-spore composition or a spore composition comprises one or more of B. subtilis, licheniformis, pumilus, megaterium, simplex, and amyloliquefaciens, preferably all of these species. Any combination of these ingredients, or the ingredients described with other preferred embodiments, may be used with preferred embodiments of a nutrient-spore composition, a nutrient-germinant composition, or a spore composition, unless a specific combination of ingredients is expressly excluded herein.

The preferred Bacillus spores for use in making a bioaugmentation solution comprises one or more of the following species Bacillus licheniformis, Bacillus subtilis, Bacillus amyloliquiefaciens, Bacillus polymyxa, Bacillus thuringiensis, Bacillus megaterium, Bacillus coagulans, Bacillus lentus, Bacillus clausii, Bacillus circulans, Bacillus firmus, Bacillus lactis, Bacillus laterosporus, Bacillus laevolacticus, Bacillus polymyxa, Bacillus pumilus, Bacillus simplex, and Bacillus sphaericus. Other Bacillus spore species may also be used as will be understood by those of ordinary skill in the art.

In one preferred embodiment for a bioaugmentation solution the method for use in an anaerobic digester, the Bacillus are preferably anaerobic bacteria or facultative anaerobes, including one or more of the following species: algicola, amyloliquefaciens, arseniciselenatis, barbaricus, circulans, coagulans, firmus, jeotgali, krulwichiae, licheniformis, mycoides, mojavensis, nealsonii, novalis, pseudomycoides, safensis, simplex, smithii, sonorensis, subtilis, thermoamylovorans, vedderi, and vallismortis. According to another preferred embodiment for use with digester systems with low oxygen concentrations (not fully anaerobic), preferred Bacillus spores include one or more of the following: clausii, lactis, laterosporus, laevolacticus, lentus, polymyxa, pumilus, megaterium, sphaericus, and toyonensis. Most preferably, 3 to 12 Bacillus species, more preferably 5 to 10 Bacillus species, and most preferably 6 to 8 Bacillus species are used in making a bioaugmentation solution. Preferred combinations include, but are not limited to: (1) licheniformis, pumilus, subtilis, and megaterium; or (2) licheniformis, pumilus, subtilis, amyloliquefaciens, and simplex; or (3) licheniformis, pumilus, subtilis, amyloliquefaciens, simplex, and megaterium; or (4) licheniformis, pumilus, subtilis, amyloliquefaciens, simplex, megaterium, and toyonensis.

Most preferably, the Bacillus species are in a spore composition that is added to a nutrient-germinant composition at the point-of-use/heating or premixed (at the point-of manufacture or prior to shipping) with a nutrient-germinant composition to form a premixed nutrient-spore composition. Preferred spore compositions comprise 60 to 40% spores (at a concentration of 1.5×10¹¹ to 2.5×10¹¹ CFU/g Bacillus spores, more preferably 1.8×10¹¹ to 2.2×10¹¹ CFU/g) and 40 to 60% salt (table salt) and are in a dry, powdered form. Most preferably, final spore compositions (spores and salt) comprise 1.5×10¹⁰ to 2.5×10¹⁰ CFU/g Bacillus spores, more preferably 1.8×10¹⁰ to 2.2×10¹⁰ CFU/g Bacillus spores.

In another preferred embodiment, a spore composition or a premixed nutrient-spore composition for use in wastewater point-of-use applications (such as a lift station, grease trap, or drain) comprises one or more Bacillus strains that break down common contaminants in water. These contaminants include, but are not limited to protein, starch, fat, oil, grease, sugar, cellulose, and plant material. The strains preferably produce one or more of the following enzymes: proteases to hydrolyze proteins, amylases to hydrolyze starches and other carbohydrates, lipases to hydrolyze fats, glycosidases to assist in the hydrolysis of glycosidic bonds in complex sugars and to assist in degradation of cellulose, cellulases to degrade cellulose to glucose, esterase which is a lipase-like enzyme, and xylanases that degrade xylan, a polysaccharide found in plant cell walls. Bacillus strains that produce these enzymes are well known in the art. Alternatively, these Bacillus strains may also be separately added to the nutrient-germinant composition at the point-of-use.

According to one preferred embodiment, a nutrient-spore composition in powdered form preferably comprises 10% to 90% by weight of a powdered spore composition and 90% to 10% by weight of a nutrient-germinant composition, more preferably around 30 to 60% by weight of a powdered spore composition and around 40 to 70% by weight of a nutrient-germinant composition, and most preferably around 40 to 50% by weight of a powdered spore composition and around 40 to 50% by weight of a nutrient-germinant composition. According to one preferred embodiment, a nutrient-spore composition in liquid form preferably comprises about 55-95%, more preferably 70-85%, and most preferably 74-83% water; about 7-13% of a spore composition, more preferably 9-11% of a spore composition, and most preferably 9.5-10.5% of a spore composition; and about 7-12% of a nutrient germinant composition, more preferably 8-11% of a nutrient germinant composition, and most preferably 9-10% of a nutrient germinant composition. The nutrient spore composition may be premixed prior to delivery to the point-of-use (such as at manufacturing or prior to shipping or prior to a point-of-sale) or may be formed at the point-of-use by mixing a separate nutrient-germinant composition and separate Bacillus spores (or a spore composition) in these amounts.

Most preferably, a nutrient-germinant concentrate composition (or a concentrated premixed nutrient-spore composition) according to embodiments of the invention is in concentrated form and is diluted to a working solution in water or any other appropriate diluent, preferably at the point-of-use. The dilution is preferably in a range from 0.1-10% of the concentrate, more preferably 1 to 8%, and most preferably 2 to 4% and the balance water, but other amounts may also be used. Dilution may be achieved by adding water or another diluent into a reservoir with the nutrient-spore composition (or separate spore composition and nutrient-germinant composition) in an incubation device used to heat the composition(s) to form a bioaugmentation solution. Alternatively, when a combined nutrient-spore composition is used the dilution may occur as a daily “dose” of bioaugmentation solution into a water collection system (such as an anaerobic digester, lift station, grease interceptor, or pipe), without necessarily adding any water or other diluent to a reservoir in an incubation device.

When spores are included in a premixed nutrient-spore composition, the premixed nutrient-spore composition also comprises one or more germination inhibitors and/or preservatives. Preferred germination inhibitors or preservatives include NaCl, D-alanine, acid, or preservatives. Specifically, a preferred premixed nutrient-spore composition comprises a high concentration of NaCl in the range of 29-117 g/L, more preferably 43-88 g/L, most preferably 52-71 g/L, and/or one or more chemical preservatives (such as Linguard ICP or Kathon CG (which has active ingredients comprising methyl chloro isothiazolinone, around 1.15-1.18% and methyl isothiazolinone, around 0.35-0.4%)) at a final (total) concentration of 0.8-3.3 g/L, more preferably 1.2-2.7 g/L, most preferably 1.6-2.2 g/L, and/or D-alanine (a known competitive inhibitor of germination) in the range of 8-116 g/L, more preferably 26-89 g/L, most preferably 40-50 g/L. These germination inhibitors may be combined with a low in the range of 3-5.5, more preferably, 3.5-5, most preferably 4-4.5. These germination inhibitors or preservatives maintain the spores in an inactive state and prevent premature germination of the spores prior to their dilution and activation at the point-of-use. The use of germination inhibitors is particularly preferred when the composition according to this embodiment is used with the preferred method of the invention, where germination occurs at the point-of-use.

Spore compositions for bioaugmentation according to preferred embodiments of the invention optionally comprise other standard ingredients including, but not limited to, other preservatives that ensure the shelf-life of the composition, surfactants that aid in the dispersal of active ingredients, a fragrance, and thickeners to keep spores suspended in solution, that are typically included in spore compositions or in industrial treatment products. Surfactants may include mild non-ionic surfactants (such as ethoxylated and alkoxylated fatty acids, ethoxylated amines, ethoxylated alcohol, alkyl and nonyl-phenol ethoxylates, ethoxylated sorbitan esters (i.e. Tween), and castor oil ethoxylate). Mild surfactants are preferable as they do not denature proteins and will not impede protein functions necessary for germination. Non-ionic surfactants may be included in the range of 1.5-2.5 g/L, more preferably 1.8-2.2 g/L, and most preferably 1.9-2.1 g/L. In addition, multiple surfactant types may be used including amphoteric surfactants (such as betaines and amine oxides). Amphoteric surfactants may be included in the range of 1.5-2.5 g/L, more preferably 1.8-2.2 g/L, and most preferably 1.9-2.1 g/L. A fragrance, preferably water-based, may be included in the range of 1.31-2.19 g/L, more preferably 1.58-1.93 g/L, and most preferably 1.66-1.84 g/L. A rheology modifier may be included for the purpose of maintaining the spores in solution. Thickeners may be natural (such as cellulose derivatives, Guar gum, Locust Bean gum, Xanthan gum, and gelatin) or synthetic (carbomer, acrylate copolymers, etc.) and may be included in the range of 1.88-3.625 g/L, more preferably 2.25-3.19 g/L, and most preferably 2.38-3.045 g/L. The amounts of these ingredients would be the same for use in a premixed nutrient-spore composition, with a proportional decrease in the amount of water. These ingredients are important for the stability of the pre-mixed nutrient-spore composition. For example, the surfactants act to disperse spores (which often times like to clump together), the preservative increases shelf-stability, and the thickener suspends the spores in solution so that each dose is consistent.

According to one preferred embodiment, a method of germinating spores at a point-of-use to form a bioaugmentation solution according to the invention comprises (1) providing a nutrient-spore composition (preferably a premixed nutrient-spore composition according to preferred embodiments of the invention or separately mixed nutrient-germinant composition and spores or a spore composition according to preferred embodiments of the invention, but other nutrient compositions and spores may also be used); (2) heating the nutrient-spore composition to an elevated temperature or range of temperatures at or near the point-of-use location; (3) maintaining the nutrient-spore composition at that temperature or within that range for a period of time (incubation period) to allow germination or activation of the spores and form a bioaugmentation solution or incubated bacteria solution; and (4) dispensing the bioaugmentation solution or incubated bacteria solution to the point-of-use, such as an anaerobic digester, lift station, grease interceptor, or drain.

Heating during the incubation period takes place in the presence of the nutrient-germination composition in a single step. Most preferably, the spores are not heat activated prior to heating with the nutrient-germinant composition at or near the point-of-use. Preferably, the spore composition is heated to a temperature in a range of 35-55° C., more preferably in the range of 38-50° C., and most preferably in the range of 41° C. to 44° C. Regardless of application, the incubation may be in an air incubator, a water incubator, or any other chamber that provides even, constant heat at the given temperature range (including a drainpipe to which heated water is added). For an anaerobic digester or wastewater application (such as a list station, grease interceptor, or drain), the incubation period is at least 15 minutes, more preferably at least 20 minutes to ensure that the bioaugmentation solution or incubated bacteria solution comprises primarily fully germinated spores that are delivered to the digester or wastewater being treated. The incubation period is preferably between 15-60 minutes, more preferably 15-25 minutes. Preferably at least 95%, more preferably at least 90%, and most preferably at least 80% of the bacteria in the bioaugmentation solution or incubated bacteria solution has germinated, but reaches outgrowth stage after being dosed to the water collection system/digester where there are ample nutrients to support the bacteria's growth.

According to another preferred embodiment, a method of germinating spores at a point-of-use to form a bioaugmentation solution according to the invention comprises (1) (a) heating a pre-mixed nutrient-spore composition to a temperature in a range of around 35° C. to 60° C. at or near an anaerobic digester or (b) providing a nutrient-germinant composition and bacteria separate from each other, mixing the nutrient-germinant composition and bacteria to form a mixed nutrient-spore composition and heating the on-site nutrient-spore composition or the nutrient-germinant composition to a temperature in a range of around 35° C. to 60° C. at or near an anaerobic digester; (2) maintaining the temperature of the premixed or mixed nutrient-spore composition for an incubation period of around 20 to 60 minutes to form a bioaugmentation solution; (3) dispensing a dose of the bioaugmentation solution to the digester; (4) wherein (a) the pre-mixed nutrient-spore composition comprises: (i) one or more L-amino acids, (ii) optionally a source of potassium ions, (iii) one or more industrial preservatives, (iv) one of more acids or salts of acids, and (v) bacteria; or (b) the nutrient-germinant composition comprises: (i) one or more L-amino acids, (ii) optionally, a source of potassium ions, (iii) one or more industrial preservatives or a germination inhibitor or both, and (iv) one or more buffers comprising a phosphate buffer, HEPES, Tris base, or a combination thereof; (5) wherein the bacteria comprise one or more of Bacillus algicola, Bacillus amyloliquefaciens, Bacillus arseniciselenatis, Bacillus barbaricus, Bacillus circulans, Bacillus coagulans, Bacillus firmus, Bacillus jeotgali, Bacillus krulwichiae, Bacillus licheniformis, Bacillus mycoides, Bacillus mojavensis, Bacillus nealsonii, Bacillus novalis, Bacillus pseudomycoides, Bacillus safensis, Bacillus simplex, Bacillus smithii, Bacillus sonorensis, Bacillus subtilis, Bacillus thermoamylovorans, Bacillus vedderi, Bacillus vallismortis; Bacillus clausii, Bacillus lactis, Bacillus laterosporus, Bacillus laevolacticus, Bacillus lentus, Bacillus polymyxa, Bacillus pumilus, Bacillus megaterium, Bacillus sphaericus, or Bacillus toyonensis in spore form; and (6) wherein the dose of bioaugmentation solution provides bacteria amounts of at least 1000 CFU per mL of the full volume capacity of the digester, even if the digester is not operated at the full volume capacity.

According to other preferred embodiments, the method and/or compositions further comprise one or more of the following: (1) the dose of bioaugmentation solution is dispensed to the hydrolysis stage of the digester; (2) the nutrient-germinant composition and bacteria are separate, the heating step comprises heating the nutrient-spore composition, and the mixed nutrient spore composition comprises around 6 to 10% of the nutrient-germinant composition and around 25 to 35% of the bacteria by weight of the mixed nutrient-spore composition; (3) the bacteria are in a powdered spore composition comprising 60 to 40% spores and 40 to 60% salt by weight of the spore composition; (4) the heating, maintaining, and dispensing steps are periodically repeated to form multiple doses of the bioaugmentation solution that are dispensed to the digester around once per 2 to 24 hours; (5) the digester is an anaerobic digester and the bacteria comprise one or more of Bacillus algicola, Bacillus amyloliquefaciens, Bacillus arseniciselenatis, Bacillus barbaricus, Bacillus circulans, Bacillus coagulans, Bacillus firmus, Bacillus jeotgali, Bacillus krulwichiae, Bacillus licheniformis, Bacillus mycoides, Bacillus mojavensis, Bacillus nealsonii, Bacillus novalis, Bacillus pseudomycoides, Bacillus safensis, Bacillus simplex, Bacillus smithii, Bacillus sonorensis, Bacillus subtilis, Bacillus thermoamylovorans, Bacillus vedderi, or Bacillus vallismortis in spore form, more preferably 2-8 of these bacteria species, most preferably 4-6 of these species; (6) the temperature range is around 35° C. to 60° C., more preferably around 42° C. to 45° C.; (7) the digester is at least partially aerobic and the bacteria comprise one or more of Bacillus clausii, Bacillus lactis, Bacillus laterosporus, Bacillus laevolacticus, Bacillus lentus, Bacillus polymyxa, Bacillus pumilus, Bacillus megaterium, Bacillus sphaericus, or Bacillus toyonensis in spore form more preferably 2-8 of these bacteria species, most preferably 4-6 of these species; (8) the nutrient-germinant composition is in a concentrated form and comprises: (a) around 8.9-133.5 g/L total of the one or more L-amino acids, (b) optionally around 7.4-55.8 g/L of potassium chloride; and (c) around 10-36 g/L monosodium phosphate, or around 30-90 g/L disodium phosphate, or around 15-61 g/L Tris base, or around 32.5-97.5 g/L HEPES, or a combination thereof; (9) the nutrient-germinant composition is diluted to around 4-10% with water; (10) at least 80% of the bacteria in the bioaugmentation solution are in an activated state when the bioaugmentation solution is dispensed to the digester; (11) the premixed nutrient-spore composition is in a concentrated form comprising (a) around 8.9-133.5 g/L each of the one or more L-amino acids, (b) around 0.8-3.3 g/L total of the one or more industrial preservatives, (c) around 1-5 g/L of citric acid or 0.0001-0.005 g/L of acid (e.g. HCl, phosphoric acid, etc.); (d) optionally, 10-30 g/L of a source of potassium, and (e) one or more of (i) around 29-117 g/L NaCl, (ii) around 0.8-3.3 g/L of one or more industrial preservatives, or (iii) wherein the nutrient-spore composition has a pH of around 4.5-5.5; (12) the nutrient-germinant composition further comprises a source of potassium chloride, monopotassium phosphate, dipotassium phosphate or a combination thereof; (13) the nutrient-germinant composition or premixed nutrient-spore composition does not include any (a) sources of nitrogen-hydrogen compounds, (b) chloride compounds other than sodium chloride or potassium chloride or (c) sugars.

Most preferably, the steps of forming a bioaugmentation or incubated bacteria solution are periodically repeated to form multiple doses of bioaugmentation or incubated bacteria solution that are each added to the point-of-use. For an anaerobic digester, preferably a dose of bioaugmentation solution comprises around (1) 4×10⁹ to 5×10¹⁰, more preferably 5×10⁹ to 4×10¹⁰, and most preferably 6×10⁹ to 3×10¹⁰ total CFUs per tonne of feedstock or (2) 2.7×10² to 2.3×10³ CFU/mL, more preferably around 3.2×10² to 2×10³ CFU/mL, and most preferably around 3.4×10² to 1.9×10³ CFU/mL of volume in the digester. According to another preferred embodiment, a dose of bioaugmentation solution comprises around 1.8×10³-2.4×10³ CFU/mL volume in the digester. Most preferably, a dose of bioaugmentation solution comprises these CFU/ml amounts based on the full volume capacity of the digester even if the digester is not operated at the full volume capacity. According to other preferred embodiments, these dosage amounts may be proportioned based on the actual volume at which the digester is operated. The number of doses daily will depend on the size of the digester, but is preferably around 1 to 5 doses daily. The digester in Example 1 below was a large digester showed improvements in methane production with 5 daily doses. Most preferably, each dose of bioaugmentation solution is at least 1000 CFU per mL of the full volume capacity of the digester, even if the digester is not operated at the full volume capacity.

For a grease interceptor or lift/pump station, preferably a dose of bioaugmentation solution comprises around (1) 1×10⁴ to 2×10⁵, more preferably 3×10⁴ to 1.5×10⁵, and most preferably 5×10⁴ to 1.4×10⁵ total CFUs added per mL of volume in the grease interceptor or lift/pump station or (2) 1.5×10¹¹ to 6.25×10¹¹ CFU, more preferably around 1.8×10¹¹ to 5.5×10¹¹ CFU, and most preferably around 1.9×10¹¹ to 5.25×10¹¹ CFU of Bacillus in the trap/lift station.

Various compositions according to preferred embodiments of the invention were tested according to preferred methods of the invention. The compositions, methods, and results are described below.

EXAMPLE 1—A nutrient-spore composition according to one preferred embodiment of the invention, in a low dose and a high dose, was used to treat a gas to grid anaerobic digester for the purpose of increased methane (CH₄) production. The nutrient-spore composition was mixed just prior to heating (at or near the point-of-use digester) from a nutrient-germinant composition and separate spore liquid composition, both according to preferred embodiments of the invention. The nutrient germinant was in a concentrated form and comprised approximately 89 g/L L-alanine, 44.7 g/L potassium chloride, 15 g/L of disodium phosphate, 5 g/L monosodium phosphate, and 1 g/L of an industrial preservative (Kathon CG or Linguard ICP, which both contain active ingredients comprising methyl chloro isothiazolinone and methyl isothiazolinone). The liquid spore composition comprised 100 g/L (approx. 2×10¹¹ CFUs/g) of a Bacillus spore blend (preferably 40-60% salt (table salt) and 60-40% Bacillus spores), Tween 80 (2 g/L), Amphosol CG (2 g/L, an amphoteric surfactant comprising around 30% active cocamidopropyl betaine), Keltrol (2.5 g/L), citric acid (1 g/L), and 1 g/L of Linguard ICP. The Bacillus species used were B. subtilis, licheniformis, pumilus, megaterium, simplex, and amyloliquefaciens. The concentrated nutrient germinant composition was diluted to 7.7% in water and the liquid spore composition was diluted to 30.8% to form the nutrient-spore composition. The composition of the low dose and high dose were the same, but the number of daily doses was increased to get from a low dose to a high dose. The nutrient-spore composition was heated to approx. 42° C. for at least 15 minutes. A dose of the bioaugmentation solution (either a low dose or a high dose as described below) was added into a 3 MW digester daily. Each dose was added into the feed lines of a gas to grid plant, so the bioaugmentation solution was present at the first stage of digestion. The digester feedstock was a mixed organic load (food manufacturing waste with mixed cereal grains) and methane (CH₄) gas output was measured as a function of biogas produced per ton of dry mass of feedstock (CH₄/ton of dry mass). Biogas production was compared to a predetermined baseline measurement of digester output before treatment with the bioaugmentation solution.

Using the same bioaugmentation solution having around 6.15×10⁹ CFU/mL of bioaugmentation solution, a low dose bacteria application having around 7×10²-1×10³ CFU/mL volume in the digester was tested initially and then a higher dose application having around 1.8×10³-2.4×10³ CFU/mL volume in the digester was used. The low dose treatment was 1 dose per day (1×10¹² total CFUs) and the high dose was 5 doses per day (5×10¹² total CFUs). The exact dose, in CFU/mL, is representative of the total final bacteria per unit volume of the digester after dosing, which depends on the percent of maximum capacity of the digester. For example, if you add 1.8×10³ CFU/ml to a digester that is filled to 100% capacity you would have 1800 CFU/ml, but if you add 1.8×10³ CFU/ml a digester that is at only 75% capacity you would have 2400 CFU/ml, making the bacterial dose effectively higher compared to operating at 100%. Results showed that a low daily dose had no impact on gas production and may have caused an apparent decrease in gas production, which was surprising. A high daily dose resulted in an increase in gas production of 5.3% above baseline.

| | Anaerobic Digester Trial (3MWe) | | | |
|---|---|---|---|---|
| | | Baseline | | |
| | | CH₄ | | |
| | | Production | Treated CH₄ | |
| | Days | kg/Ton | Production kg/Ton | % Change Relative |
| | Treated | Dry Mass | Dry Mass | to Baseline |
| Low Dose | 44 | 375.0 | 366.0 | −2.46 |
| High Dose | 36 | 375.0 | 396.0 | 5.30 |

These amounts are adjusted for just the CH₄ content of the gas. Normally, the plant outputs approx. 40,000 MWH annually. A 5.3% increase represents about 2,100 MWH.

The industrial digester used for the trial transfers the methane produced directly to the natural gas power grid. The gas to grid value of the digester is approx. $6.3 million in methane yearly. The increase in gas production in this trial represents about $300,000 annual increase in digester profitability. Any ingredient or method steps of a preferred embodiment herein may be used with any other ingredients, features, components or steps of other embodiments even if not specifically described with respect to that embodiment, unless such combination is explicitly excluded herein. Any ingredient or amount of an ingredient, or method steps described as excluded with any particular preferred embodiment herein may similarly be excluded with any other preferred embodiment herein even if not specifically described with such embodiment. All numerical values for amounts of ingredients, ratios, temperatures, and incubation periods, herein described as a range specifically include any individual value or ratio within such ranges and any and all subset combinations within ranges, including subsets that overlap from one preferred range to a more preferred range and even if the specific subset of the range is not specifically described herein. Those of ordinary skill in the art will also appreciate upon reading this specification and the description of preferred embodiments herein that modifications and alterations to the device may be made within the scope of the invention and it is intended that the scope of the invention disclosed herein be limited only by the broadest interpretation of the appended claims to which the inventors are legally entitled.

We claim:

1. A method of increasing methane production in a digester having a full volume capacity, the method comprising the following steps:
heating a nutrient-spore composition to a temperature in a range of around 35° C. to 60° C. at or near a digester;
maintaining the temperature of the nutrient-spore composition for an incubation period of around 20 to 60 minutes to form a bioaugmentation solution;
dispensing a dose of the bioaugmentation solution to the digester;
wherein the nutrient-spore composition comprises: (a) one or more L-amino acids, (b) one or more industrial preservatives, (c) one or more acids or salts of acids, (d) a thickener, and (e) bacteria;
wherein the bacteria comprises one or more of Bacillus algicola, Bacillus amyloliquefaciens, Bacillus arseniciselenatis, Bacillus barbaricus, Bacillus circulans, Bacillus coagulans, Bacillus firmus, Bacillus jeotgali, Bacillus krulwichiae, Bacillus licheniformis, Bacillus mycoides, Bacillus mojavensis, Bacillus nealsonii, Bacillus novalis, Bacillus pseudomycoides, Bacillus safensis, Bacillus simplex, Bacillus smithii, Bacillus sonorensis, Bacillus subtilis, Bacillus thermoamylovorans, Bacillus vedderi, Bacillus vallismortis; Bacillus clausii, Bacillus lactis, Bacillus laterosporus, Bacillus laevolacticus, Bacillus lentus, Bacillus polymyxa, Bacillus pumilus, Bacillus megaterium, Bacillus sphaericus, or Bacillus toyonensis in spore form; and
wherein the dose of bioaugmentation solution provides bacteria amounts of at least 1000 CFU per mL of the full volume capacity of the digester, even if the digester is operated at less than the full volume capacity.

2. The method of claim 1 wherein the dose of bioaugmentation solution is dispensed to a hydrolysis stage of the digester.

3. The method of claim 1 wherein the nutrient-spore composition comprises around 6 to 10% by weight of a nutrient-germinant composition and around 25 to 35% by weight of a spore composition, the method further comprising mixing the nutrient-germinant composition and the spore composition to form the nutrient-spore composition prior to or during the heating step; and
wherein the nutrient-germinant composition comprises (1) around 8.9-133.5 g/L total of the one or more L-amino acids; (2) optionally around 7.4-55.8 g/L of potassium chloride; and (3) around 10-36 g/L monosodium phosphate, or around 30-90 g/L disodium phosphate, or around 15-61 g/L Tris base, or around 32.5-97.5 g/L HEPES, or a combination thereof; and
wherein the spore composition is a liquid comprising (1) the one or more acids or salts of acids, (2) the thickener, and (3) a spore blend.

4. The method of claim 3 wherein the spore blend is in a powdered form comprising 60 to 40% of the bacteria and 40 to 60% salt by weight of the spore blend.

5. The method of claim 4 wherein the one or more acids or salts of acids comprises citric acid and wherein spore composition further comprises (1) around 75-125 g/L of the spore blend, (2) around 1.5-2.5 g/L of a first surfactant, (3) around 1.5-2.5 g/L of a second surfactant, (4) around 2.175-3.625 g/L of the thickener, (5) around 0.75-1.25 g/L of the citric acid, and (6) around 0.75-1.25 g/L of the one or more industrial preservatives.

6. The method of claim 5 wherein the spore blend comprises at least around 2×10¹¹ CFUs/g of the bacteria.

7. The method of claim 5 wherein the first surfactant is polysorbate 80 and the second surfactant is an amphoteric surfactant comprising cocamidopropyl betaine.

8. The method of claim 7 wherein the thickener comprises xanthan gum.

9. The method of claim 3 wherein the dose of bioaugmentation solution is dispensed to the hydrolysis stage of the digester and wherein the heating, maintaining, and dispensing steps are periodically repeated to form multiple doses of the bioaugmentation solution that are dispensed to the digester around once per 2 to 24 hours.

10. The method of claim 9 wherein the digester is an anaerobic digester and the bacteria comprise one or more of Bacillus algicola, Bacillus amyloliquefaciens, Bacillus arseniciselenatis, Bacillus barbaricus, Bacillus circulans, Bacillus coagulans, Bacillus firmus, Bacillus jeotgali, Bacillus krulwichiae, Bacillus licheniformis, Bacillus mycoides, Bacillus mojavensis, Bacillus nealsonii, Bacillus novalis, Bacillus pseudomycoides, Bacillus safensis, Bacillus simplex, Bacillus smithii, Bacillus sonorensis, Bacillus subtilis, Bacillus thermoamylovorans, Bacillus vedderi, or Bacillus vallismortis in spore form and
wherein the temperature range is around 38° C. to 50° C.

11. The method of claim 9 wherein the digester is at least partially aerobic and the bacteria comprise one or more of Bacillus clausii, Bacillus lactis, Bacillus laterosporus, Bacillus laevolacticus, Bacillus lentus, Bacillus polymyxa, Bacillus pumilus, Bacillus megaterium, Bacillus sphaericus, or Bacillus toyonensis in spore form.

12. The method of claim 3 wherein the nutrient-germinant composition is in a concentrated form and wherein the method further comprises diluting the nutrient-germinant composition to around 4-10% with water prior to or during the mixing step.

13. The method of claim 1 wherein at least 80% of the bacteria in the bioaugmentation solution are in an activated state when the bioaugmentation solution is dispensed to the digester.

14. The method of claim 1 wherein the one or more acids or salts of acids comprises citric acid; and
wherein the nutrient-spore composition is in a concentrated form comprising (1) around 8.9-133.5 g/L each of the one or more L-amino acids, (2) around 0.8-3.3 g/L total of the one or more industrial preservatives, (3) around 1-5 g/L of the citric acid, (4) 10-30 g/L of a source of potassium, and (5) one or both of (a) around 29-117 g/L NaCl or (b) wherein the nutrient-spore composition has a pH of around 4.5-5.5.

15. The method of claim 1 wherein the nutrient-spore composition further comprises a source of potassium chloride, monopotassium phosphate, dipotassium phosphate or a combination thereof.

16. The method of claim 1 wherein the nutrient-spore composition (1) does not include any sources of nitrogen-hydrogen compounds, (2) does not include any chloride compounds other than sodium chloride or potassium chloride and (3) does not include any sugars.

17. The method of claim 16 wherein the digester is an anaerobic digester and the bacteria comprise one or more of Bacillus subtilis, Bacillus licheniformis, Bacillus pumilus, Bacillus megaterium, Bacillus simplex, and Bacillus amyloliquefaciens.

18. The method of claim 5 wherein at least 80% of the bacteria in the bioaugmentation solution are in an activated state when the bioaugmentation solution is dispensed to the digester;
wherein the dose of bioaugmentation solution is dispensed to the hydrolysis stage of the digester;
wherein the nutrient-spore composition does not include any (1) sources of nitrogen-hydrogen compounds, (2) chloride compounds other than sodium chloride or potassium chloride or (3) sugars; and
wherein the digester is an anaerobic digester and the bacteria comprise one or more of Bacillus subtilis, Bacillus licheniformis, Bacillus pumilus, Bacillus megaterium, Bacillus simplex, and Bacillus amyloliquefaciens.

19. The method of claim 5 wherein the nutrient-spore composition does not include any sources of nitrogen-hydrogen compounds.

20. The method of claim 5 wherein the nutrient-spore composition does not include any chloride compounds other than sodium chloride or potassium chloride.

21. The method of claim 5 wherein the nutrient-spore composition does not include any sugars.
